(19) United States
(12) Patent Application Publication
(10) Pub. No.: US 2026/0174706 A1
(43) Pub. Date: Jun. 25, 2026

(54) LIPID NANOPARTICLE FORMULATION FOR TRANSPULMONARY DELIVERY OF NUCLEIC ACID DRUGS AND USE THEREOF

(71) Applicant: EWHA UNIVERSITY - INDUSTRY COLLABORATION FOUNDATION, Seoul (KR)

(72) Inventors: Hyukjin LEE, Seoul (KR); Yun-Sil LEE, Seoul (KR); Hee JIN, Seoul (KR); Michaela JEONG, Seoul (KR); Yeji LEE, Gyeonggi-do (KR)

(73) Assignee: EWHA UNIVERSITY - INDUSTRY COLLABORATION FOUNDATION, Seoul (KR)

(21) Appl. No.: 19/126,451

(22) PCT Filed: Oct. 31, 2023
(86) PCT No.: PCT/KR2023/017108

§ 371 Date: May 1, 2025

(30) Foreign Application Priority Data
Nov. 2, 2022 (KR) 10-2022-0144737
Sep. 4, 2023 (KR) 10-2023-0117261

(51) Int. Cl. A61K 9/51; A61K 9/00; A61K 31/7105; A61P 11/00

(52) U.S. Cl. CPC A61K 9/5146; A61K 9/007; A61K 9/5123; A61K 31/7105; A61P 11/00

(57) ABSTRACT

The present invention relates to a composition for transpulmonary delivery of nucleic acid drugs and a use thereof. The composition for transpulmonary delivery of the present invention is specific to lung tissue and specific cells thereof, has excellent biocompatibility, and can deliver gene therapy products, etc., with high efficiency, and thus can be useful in relevant technical fields such as in the treatment of lipid nanoparticle-mediated pulmonary diseases.

### TECHNICAL FIELD

The present invention relates to a composition for transpulmonary delivery of nucleic acid drugs and use thereof.

### BACKGROUND ART

Pulmonary fibrosis (lung fibrosis) is an irreversible chronic disease based on excessive production of extracellular matrix caused by overexpressed inflammatory activity in environmentally or chemically damaged lung tissue. Abnormal accumulation of fibrous tissue leads to airway narrowing and thickens the epithelium in the alveoli, impairing the blood's supply of oxygen and causing serious respiratory dysfunction.

The incidence of pulmonary fibrosis is rising, but current treatments are very limited, and there is an urgent need to elucidate its pathophysiological mechanisms and develop new treatments. Recently, Gtse1 (G2 and S-phase expressed 1) was identified as being associated with pulmonary fibrosis in a radiation-induced pulmonary fibrosis model, implying that Gtse1 and its downstream genes may be potential targets for the treatment of the disease (H Jin et al., J Mol Med., 2019, January, 97(1):37-47).

Nucleic acids, such as antisense RNA and siRNA, are capable of inhibiting the expression of specific proteins in vivo and have emerged as important tools in the treatment of cancer, genetic diseases, infectious diseases, and autoimmune disorders (Novina and Sharp, Nature, 430, 161-164, 2004). However, nucleic acids such as siRNA are difficult to deliver directly into cells and are easily degraded by enzymes in the blood, and thus extensive research is underway to develop strategies to overcome these problems.

A drug delivery system (DDS) is a technology engineered to deliver the necessary dosage of a drug effectively, minimizing side effects while maximizing efficacy and effectiveness. In particular, the conventional viral delivery systems have been proven to be effective as drug delivery vehicles in gene therapy, but several drawbacks such as immunogenicity, limitations in the size of injected DNA, and difficulties in mass production have limited the use of viruses as gene delivery systems.

Therefore, as an alternative to viral systems, a method of transporting nucleic acids into cells has been mainly used so far by mixing nucleic acids with positively charged lipids or polymers (named lipid-DNA conjugates (lipoplexes) and polymer-DNA conjugates (polyplexes), respectively) (Hirko et al., Curr, Med, Chem., 10, 1185-1193, 2003; Merdan et al., Adv. Drug. Deliv. Rev., 54, 715-758, 2002; Spagnou et al., Biochemistry, 43, 13348-13386, 2004). In particular, the lipid-DNA conjugates are widely used at the cellular level because of their ability to bind to nucleic acids and deliver the nucleic acids well into the cell, but in an in vivo environment, there are some disadvantages that the conjugates often cause inflammation in the body when injected locally (Filonand and Phillips, Biochim. Biophys/Acta, 1329, 345-356, 1997), and when injected intravascularly, the conjugates accumulate in tissues such as the lung, liver, and spleen, which are primarily first-pass organs (Ren et al., Gene Therapy. 7, 764-768, 2000).

In light of these challenges, the present inventors have made extensive efforts to develop a carrier capable of efficiently delivering anionic drugs such as nucleic acids to lung organs or cells to treat pulmonary fibrosis, etc., and have thus completed the present invention by confirming the superior drug delivery effect of a mannose-containing lipid nanoparticle formulation of the present invention.

### DISCLOSURE

### Technical Problem

An object of the present invention is to provide a composition for transpulmonary delivery of a drug comprising lipid nanoparticles containing a mannose-polyethyleneglycol (PEG)-lipid conjugate.

Another object of the present invention is to provide a pharmaceutical composition for preventing or treating pulmonary diseases, comprising the lipid nanoparticles and an anionic drug.

### Technical Solution

The present disclosure will be described in detail as follows. Meanwhile, each description and embodiment disclosed in the present disclosure may be applied to each of the other descriptions and embodiments. In other words, all combinations of various elements disclosed in the present disclosure fall within the scope of the present disclosure. In addition, it cannot be considered that the scope of the present disclosure is limited by specific descriptions described below.

In an embodiment for achieving the above object, the present invention provides a composition for transpulmonary delivery of a drug comprising lipid nanoparticles containing a mannose-polyethyleneglycol (PEG)-lipid conjugate.

The lipid nanoparticles containing the mannose-PEG-lipid conjugate of the present invention may be specifically delivered to lung tissue, specifically, fibrotic lungs or cells thereof (e.g., lung epithelial cells, endothelial cells, etc.), and thus anionic drugs, etc., are able to be specifically delivered to fibrotic lungs with high efficiency. In other words, the lipid nanoparticle of the present invention may specifically target fibrotic lung tissue, and specifically target lung epithelial cells and/or endothelial cells of fibrotic lung tissue.

The term “targeting” as used herein may refer to internalization within a tissue or a cell, and may also refer to mean internalization within the nucleus by penetrating the nuclear membrane.

The composition of the present invention may be locally delivered within the lung, and specifically may be locally delivered to fibrotic lung.

The term “mannose-PEG-lipid conjugate” as used herein refers to a conjugate of mannose, polyethyleneglycol (PEG), and a lipid, and may refer to a lipid having mannose conjugated to one end of the lipid and PEG conjugated to the other end of the lipid.

The mannose-PEG-lipid conjugate may form a mixture of PEG-lipid conjugates together with other PEG-lipids, wherein “PEG-lipid conjugate mixture” may mean a mixture comprising one or more other PEG-lipid conjugates together with the mannose-PEG-lipid conjugate. The PEG-lipid may be, for example, but not limited to, PEG-ceramide, PEG-DMG, PEG-c-DOMG, PEG-DLPE, PEG-DMPE, PEG-DPPC, PEG-DSPE or a mixture thereof.

The PEG-lipid conjugate mixture may contribute to the stability of the nanoparticles in serum within the lipid nanoparticles, and may play a role in preventing aggregation between nanoparticles, and increasing the targeting effect to lung tissue or specific cells thereof. In addition, the PEG-lipid conjugate mixture may protect anionic drugs such as nucleic acids from degradative enzymes during in vivo delivery, thereby enhancing the in vivo stability of drugs and increasing the half-life of drugs encapsulated in nanoparticles.

In the PEG-lipid conjugate or mannose-PEG-lipid conjugate, PEG or mannose may be directly conjugated to the lipid or linked to the lipid via a linker moiety. Any linker moiety suitable for conjugating PEG to the lipid may be used, including, for example, ester-free linker moieties and ester-containing linker moieties. The ester-free linker moieties may include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl NHC(O)CH₂CH₂C(O)NH—), ether, disulfide, as well as combinations thereof (e.g., linkers containing both a carbamate linker moiety and an amido linker moiety). The ester-containing linker moieties may include, but are not limited to, for example, carbonate (—OC(O)O—), succinoyl, phosphate ester (—O—(O)POH—O—), sulfonate ester, and combinations thereof.

The PEG-lipid conjugate mixture (e.g., mannose-PEG lipid conjugate and/or PEG-ceramide conjugate) may have an average molecular weight of 100 to 10,000 daltons, 200 to 10,000 daltons, 500 to 10,000 daltons, 1,000 to 10,000 daltons, 1,500 to 10,000 daltons, 2,000 to 10,000 daltons, 100 to 7,500 daltons, 200 to 7,500 daltons, 500 to 7,500 daltons, 1,000 to 7,500 daltons, 1,500 to 7,500 daltons, 2,000 to 7,500 daltons, 100 to 5,000 daltons, 200 to 5,000 daltons, 500 to 5,000 daltons, 1,000 to 5,000 daltons, 1,500 to 5,000 daltons, 2,000 to 5,000 daltons, 100 to 3,000 daltons, 200 to 3,000 daltons, 500 to 3,000 daltons, 1,000 to 3,000 daltons, 1,500 to 3,000 daltons, 2,000 to 3,000 daltons, 100 to 2,600 daltons, 200 to 2,600 daltons, 500 to 2,600 daltons, 1,000 to 2,600 daltons, 1,500 to 2,600 daltons, 2,000 to 2,600 daltons, 100 to 2,500 daltons, 200 to 2,500 daltons, 500 to 2,500 daltons, 1,000 to 2,500 daltons, 1,500 to 2,500 daltons, or 2,000 to 2,500 daltons.

The lipid in the PEG-lipid conjugate may be used without limitation as long as it is any lipid capable of conjugating to polyethylene glycol, and specifically may be, but is not limited to, ceramide, dimyristoylglycerol (DMG), succinoyl-diacylglycerol (s-DAG), distearoylphosphatidylcholine (DSPC), distearoylphosphatidylethanolamine (DSPE), or cholesterol.

The PEG-lipid conjugate mixture may comprise, in addition to the mannose-PEG-lipid conjugate, PEG conjugated to dialkyloxypropyl (PEG-DAA), PEG conjugated to diacylglycerol (PEG-DAG), PEG conjugated to phospholipids such as phosphatidylethanolamine (PEG-PE), PEG conjugated to ceramide (PEG-CER, or ceramide-PEG conjugate), PEG conjugated to cholesterol or a derivative thereof, PEG-c-DOMG, PEG-DMG, PEG-DLPE, PEG-DMPE, PEG-DPPC, PEG-DSPE, and mixtures thereof, specifically, C16-PEG2000 ceramide, DMG-PEG 2000, 14:0 PEG2000 P E. According to an embodiment of the present invention, the PEG-lipid conjugate mixture may comprise a PEG-ceramide conjugate and/or a mannose-PEG-lipid conjugate (e.g., a mannose-PEG-DSPE conjugate).

The PEG-lipid conjugate mixture may be contained in the lipid nanoparticles at 0.1 to 15 mol %, 0.25 to 15 mol %, 0.5 to 15 mol %, 1 to 15 mol %, 1.5 to 15 mol %, 2 to 15 mol %, 2.5 to 15 mol %, 3 to 15 mol %, 0.1 to 10 mol %, 0.25 to 10 mol %, 0.5 to 10 mol %, 1 to 10 mol %, 1.5 to 10 mol %, 2 to 10 mol %, 2.5 to 10 mol %, 3 to 10 mol %, 0.1 to 9 mol %, 0.25 to 9 mol %, 0.5 to 9 mol %, 1 to 9 mol %, 1.5 to 9 mol %, 2 to 9 mol %, 2.5 to 9 mol %, 3 to 9 mol %, 0.1 to 7.5 mol %, 0.25 to 7.5 mol %, 0.5 to 7.5 mol %, 1 to 7.5 mol %, 1.5 to 7.5 mol %, 2 to 7.5 mol %, 2.5 to 7.5 mol %, 3 to 7.5 mol %, 0.1 to 5 mol %, 0.25 to 5 mol %, 0.5 to 5 mol %, 1 to 5 mol %, 1.5 to 5 mol %, 2 to 5 mol %, 2.5 to 5 mol %, 3 to 5 mol %, 0.1 to 4.5 mol %, 0.25 to 4.5 mol %, 0.5 to 4.5 mol %, 1 to 4.5 mol %, 1.5 to 4.5 mol %, 2 to 4.5 mol %, 2.5 to 4.5 mol %, 3 to 4.5 mol %, 0.1 to 4 mol %, 0.25 to 4 mol %, 0.5 to 4 mol %, 1 to 4 mol %, 1.5 to 4 mol %, 2 to 4 mol %, 2.5 to 4 mol %, 3 to 4 mol %, 0.1 to 3.5 mol %, 0.25 to 3.5 mol %, 0.5 to 3.5 mol %, 1 to 3.5 mol %, 1.5 to 3.5 mol %, 2 to 3.5 mol %, 2.5 to 3.5 mol %, 3 to 3.5 mol %, 0.1 to 3 mol %, 0.25 to 3 mol %, 0.5 to 3 mol %, 1 to 3 mol %, 1.5 to 3 mol %, 2 to 3 mol %, or 2.5 to 3 mol %.

The mannose-PEG-lipid conjugate may be contained in the lipid nanoparticles at 0.1 to 10 mol %, 0.25 to 10 mol %, 0.5 to 10 mol %, 1 to 10 mol %, 1.5 to 10 mol %, 2 to 10 mol %, 0.1 to 7.5 mol %, 0.25 to 7.5 mol %, 0.5 to 7.5 mol %, 1 to 7.5 mol %, 1.5 to 7.5 mol %, 2 to 7.5 mol %, 0.1 to 5 mol %, 0.25 to 5 mol %, 0.5 to 5 mol %, 1 to 5 mol %, 1.5 to 5 mol %, 2 to 5 mol %, 0.1 to 4.5 mol %, 0.25 to 4.5 mol %, 0.5 to 4.5 mol %, 1 to 4.5 mol %, 1.5 to 4.5 mol %, 2 to 4.5 mol %, 0.1 to 4 mol %, 0.25 to 4 mol %, 0.5 to 4 mol %, 1 to 4 mol %, 1.5 to 4 mol %, 2 to 4 mol %, 0.1 to 3 mol %, 0.25 to 3 mol %, 0.5 to 3 mol %, 1 to 3 mol %, 1.5 to 3 mol %, 2 to 3 mol %, 0.1 to 2.5 mol %, 0.25 to 2.5 mol %, 0.5 to 2.5 mol %, 1 to 2.5 mol %, 1.5 to 2.5 mol %, 2 to 2.5 mol %, 0.1 to 2 mol %, 0.25 to 2 mol %, 0.5 to 2 mol %, 1 to 2 mol %, or 1.5 to 2 mol %.

The lipid nanoparticles containing the mannose-PEG-lipid conjugate of the present invention may further comprise at least one selected from the group consisting of ionizable lipids, phospholipids, and structural lipids.

As used herein, the term “ionizable lipid” may refer to an amine-containing lipid capable of being easily protonated, and may be, for example, a lipid whose charge state changes depending on the surrounding pH. The ionizable lipid may be positively charged at a surrounding pH below pKa and may be progressively neutral at a surrounding pH above pKa. When positively charged, the ionizable lipid may associate with a negatively charged drug (e.g., an anionic drug and/or nucleic acid), and may serve to encapsulate the drug within the lipid nanoparticle with high efficiency through electrostatic interaction with the drug.

The ionizable lipid may be any known lipid that possesses a net positive charge at a selective pH such as physiological pH without limitation, and may be 9-heptadecanyl 8-{(2-hydroxyethyl) [6-oxo-6-(undecyloxy)hexyl]amino}octanoate (SM-102), [(4-hydroxybutyl)azanediyl]di(hexane-6,1-diyl)bis(2-hexyldecanoate) (ALC-0315), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N-(N′,N′-dimethylaminoethane)-carbamoyl) cholesterol (DC-Chol), N-(1,2-dimyristyloxyprop-3-yl)-N,N-dimethyl-N-hydroxyethylammonium bromide (DMRIE), 2,3-dioleyloxy-N-[2-(spermine-carboxamido)ethyl]-N,N-dimethyl-1-propanaminium trifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3R-oxy)-3′-oxapentoxy]-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 1,2-dilinoleyloxy-N,N-dimethylaminopropane (DLinDMA), 1,2-dilinolenyloxy-N,N-dimethylaminopropane (DLenDMA), heptatriacont-6,9,28,31-tetraen-19-yl 4-(dimethylamino)butanoate (DLin-MC3-DMA), 1, 1′,1″,1″-[1,4-piperazinediylbis (3,1-propanediylnitrilo)]tetrakis-2-dodecanol (246-C10), (Z)-non-2-en-1-yl 9,9′-((2-(4-(9-(((Z)-non-2-en-1-yl)oxy)-9-oxononyl)piperazin-1-yl)ethyl)azanediyl)dinonanoate (244-cis) or a combination thereof.

The phospholipid may be any phospholipid capable of promoting the fusion of lipid nanoparticles without limitation, and for example, may be dioleoylphosphatidylethanolamine (DOPE), distearoylphosphatidylcholine (DSPC), palmitoyloleoylphosphatidylcholine (POPC), egg phosphatidylcholine (EPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), 1,2-dioleoyl-3-trimethylammonium-propane (DOTAP), phosphatidylethanolamine (PE), dipalmitoylphosphatidylethanolamine, 1,2-dioleoyl-sn-glycero-3-phosphate (18-PA), 1,2-dilinoleoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16:0 PE), 1-palmitoyl-2-oleoyl-sn-glycero-3-phosphoethanolamine (POPE), 1,2-dioleoyl-sn-glycero-3-[phospho-L-serine] (DOPS), 1,2-dilinoleoyl-sn-glycero-3-phosphocholine (DLPC), 1,2-dimyristoyl-sn-glycero-3-phosphocholine (DMPC), 1,2-diundecanoyl-sn-glycero-phosphocholine (DUPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-distearoyl-sn-glycero-3-phosphoethanolamine, 1,2-dilinoleoyl-sn-glycero-3-phosphoethanolamine, 1,2-dilinoleoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, sphingomyelin, or a mixture thereof.

The structural lipid serves to maintain the particle shape within the lipid nanoparticles and to improve the stability of the nanoparticles by being dispersed in the core and surface of the nanoparticles. The structural lipid may be, for example, but not limited to, cholesterol, cholestenol, spinasterol, fecosterol, sitosterol, ergosterol, ergostenol, campesterol, stigmasterol, brassicasterol, tomatidine, ursolic acid, alpha-tocopherol, or a mixture thereof.

According to an embodiment, when lipid nanoparticles are manufactured by mixing the ionizable lipid of the present invention with phospholipid, structural lipid, and PEG-lipid, a molar ratio of ionizable lipid:phospholipid:structural lipid:PEG-lipid conjugate mixture may be 20 to 60:10 to 30 to 60:0.1 to 10. Specifically, the molar ratio may be, but is not limited to, 30 to 50:10 to 20:35 to 50 0.5 to 8, and more specifically, 40 to 45:10 to 15:38 to 45:1 to 6. Among the components contained in said lipid nanoparticles, the molar ratio of the components may be maintained by keeping the sum of the moles of the PEG-lipid conjugate mixture and the structural lipid at a constant level and by decreasing the molar number of structural lipids to an extent that the molar number of the PEG-lipid conjugate mixture is increased.

The lipid nanoparticles of the present invention exhibit a positive charge under acidic pH conditions, and thus the lipid nanoparticles are able to easily form drug complexes through electrostatic interaction with therapeutic agents such as nucleic acids and anionic drugs, which exhibit a negative charge, thereby encapsulating the anionic drugs with high efficiency and being usable as intracellular or in vivo drug delivery composition. Thus, the lipid nanoparticles of the present invention may be useful for the delivery of not only nucleic acids, but also any anionic form of drug. In other words, the lipid nanoparticle of the present invention may be ultimately manufactured in a form (encapsulated form) that additionally contains an anionic drug, and the encapsulated anionic drug may be specifically delivered to the lungs, specifically, to fibrotic lungs.

As used herein, the term “encapsulation” refers to a process of encapsulating a delivery substance for surrounding and embedding it in vivo efficiently, and the drug encapsulation efficiency (encapsulation efficiency) means the amount of drug encapsulated within lipid nanoparticles relative to the total drug amount used in the preparation.

The anionic drug may be a nucleic acid, a small molecule compound, a peptide, a protein, a protein-nucleic acid construct, or an anionic biopolymer-drug conjugate, but is not limited thereto, as long as the drug is able to be stably and efficiently delivered by forming the lipid nanoparticles with the ionizable lipid of the present invention.

In the present invention, the nucleic acid may be small interfering ribonucleic acid (siRNA), ribosomal ribonucleic acid (rRNA), deoxyribonucleic acid (DNA), complementary deoxyribonucleic acid (cDNA), aptamer, messenger ribonucleic acid (mRNA), transfer ribonucleic acid (tRNA), sgRNA, antisense oligonucleotide, shRNA, miRNA, ribozyme, PNA, and DNAzyme, or a mixture thereof, and specifically, may be at least one selected from the group consisting of siRNA, mRNA, antisense oligonucleotide, and miRNA, but is not limited thereto. According to an embodiment of the present invention, the nucleic acid drug may be siGtse1, which is an siRNA that inhibits the expression of Gtse1 protein.

A weight ratio of the total lipid/nucleic acid in the lipid nanoparticle may be 1 to 20, specifically 5 to 15, more specifically 7 to 12, but is not limited thereto.

In the present invention, the lipid nanoparticle may have a diameter size of, for example, 40 to 150 nm, specifically 50 to 140 nm, and more specifically 60 to 130 nm, but is not limited thereto.

In another embodiment for achieving the above object, the present invention provides a pharmaceutical composition for preventing or treating pulmonary diseases comprising the lipid nanoparticles and an anionic drug.

In still another embodiment, the present invention provides a method of treating pulmonary diseases, comprising administering the composition to a subject in need thereof.

In still another embodiment, the present invention provides a pharmaceutical composition for use in the prevention or treatment of pulmonary diseases, or use of the composition for the prevention or treatment of pulmonary diseases.

The lipid nanoparticles and anionic drugs are described above.

The lipid nanoparticles of the present invention are effective for delivering anionic drugs due to their characteristics of forming stable complexes with anionic drugs such as nucleic acids and exhibiting low cytotoxicity and effective cellular uptake. Further, since the lipid nanoparticles may be specifically delivered to the lungs, specifically to fibrotic lungs or specific cells thereof, when administered, the lipid nanoparticles may be effectively utilized for the prevention or treatment of pulmonary diseases.

The lipid nanoparticles of the present invention may target the lungs and specifically deliver drugs to the lungs. According to an embodiment of the present invention, the lipid nanoparticles of the present invention may more specifically deliver drugs to fibrotic lungs and cells thereof (e.g., pulmonary epithelial cells and endothelial cells). Therefore, the pulmonary disease may specifically be pulmonary fibrosis.

In the present invention, the term “treatment” refers to intervention aimed at altering the natural processes of individuals or cells having a disease, which may be performed either during the progression of pathological conditions or for prevention thereof. The intended therapeutic effect includes preventing the onset or recurrence of the disease, relieving symptoms, reducing any direct or indirect pathologic consequences of the disease, preventing metastasis, slowing the rate of disease progression, alleviating or temporarily relieving a disease condition, achieving remission, or improving prognosis. In particular, the present invention encompasses any act of ameliorating the course of a pulmonary related disease by the administration of the composition comprising the lipid nanoparticles containing the mannose-PEG-lipid conjugate and an anionic drug as an active ingredient. Further, the term “prevention” refers to any act of inhibiting or delaying the onset of a disease by the administration of the lipid nanoparticles. When the lipid nanoparticles of the present invention are used for treatment or prevention purposes, the lipid nanoparticles are administered to an individual in a therapeutically effective amount.

As used in the present invention, the term “therapeutically effective amount” refers to an effective amount of anionic drug-containing lipid nanoparticles. Specifically, the “therapeutically effective amount” means an amount sufficient to treat a disease at a reasonable benefit/risk ratio applicable to medical treatment, and the effective dose level may be determined depending on factors including the subject type and severity, age, sex, type of diseases, the activity of the drug, the sensitivity to the drug, the time of administration, the route of administration, the rate of excretion, the duration of treatment, drugs used concurrently, and other factors well known in the medical field. The pharmaceutical composition of the present invention may be administered as an individual therapeutic agent or in combination with other therapeutic agents, or may be administered sequentially or simultaneously with a commercially available therapeutic agent. In addition, the pharmaceutical composition of the present invention may be administered singly or in multiple doses. In consideration of all of the above factors, it is important to administer an amount capable of obtaining the maximum effect with the minimum amount without side effects, which may be easily determined by those skilled in the art. The administration dose of the pharmaceutical composition of the present invention may be determined by specialists according to various factors such as the patient's condition, age, sex, complications, and the like. Since the active ingredient of the pharmaceutical composition of the present invention has excellent safety, the active ingredient may be used even above the predetermined dose.

The composition comprising the lipid nanoparticles may be administered through various routes, including oral, intramuscular, intravenous, intraarterial, subcutaneous, intraperitoneal, pulmonary, and intranasal injection, and specifically may be directly and locally administered into the lungs, and more specifically may be locally administered to fibrotic lungs.

The composition of the present invention may further comprise one or more additional pharmaceutically acceptable carriers for administration. The pharmaceutically acceptable carrier may be saline, sterile water, Ringer's solution, buffered saline, dextrose solution, maltodextrin solution, glycerol, ethanol, and a mixture of one or more of these components, and, if necessary, may contain other conventional additives such as antioxidants, buffers, bacteriostatic agents, and the like.

### Advantageous Effects

The lipid nanoparticles of the present invention are specific to lung tissue and specific cells thereof, have excellent biocompatibility, and can deliver gene therapy products, etc., with high efficiency, and thus can be effectively utilized in relevant technical fields such as in the treatment of lipid nanoparticle-mediated gene therapy.

### DESCRIPTION OF DRAWINGS

FIG. 1 is a schematic diagram showing an exemplary structure of mannose-modified lipid nanoparticles and its mechanism of action for pulmonary disease using the same.

FIG. 2 shows the intracellular luminescence intensity of mannose-modified lipid nanoparticles encapsulating mRNA encoding luciferase measured by using various types of ionizable lipids.

FIG. 3 is an ex vivo organ image showing bioluminescence to confirm the specific delivery of mannose-modified lipid nanoparticles in vivo.

FIG. 4 is an ex vivo organ image showing bioluminescence of lipid nanoparticles not modified with mannose.

FIG. 5 is a graph showing comparison of the fluorescence expression per lung cell of mannose-modified lipid nanoparticles in LSL-tdTomato mice (blank: normal model, colored: pulmonary fibrosis model).

FIG. 6 is a graph confirming the cytotoxicity of mannose-modified lipid nanoparticles using the CellTiter-Fluor™ Cell Viability Assay.

FIG. 7 is a graph showing the levels of aspartate aminotransferase (AST), alanine aminotransferase (ALT), blood urea nitrogen (BUN), and Creatinine measured to evaluate the presence of liver and kidney toxicity following the administration of mannose-modified lipid nanoparticles.

FIG. 8 is a graph showing the levels of glutamic oxaloacetic transaminase (GOT), glutamic pyruvic transaminase (GPT), blood urea nitrogen (BUN), and creatinine (CRE) measured to evaluate liver and kidney damage following the administration of mannose-modified lipid nanoparticles using the bleomycin-induced pulmonary fibrosis model.

FIGS. 9 and 10 are images of histopathological changes in the spleen, liver, kidney, and lungs observed by hematoxylin-eosin (H&E) staining to confirm histopathological changes in major organs following the administration of mannose-modified lipid nanoparticles.

FIG. 11 shows the inhibitory effect of mannose-modified lipid nanoparticles on the Gtse1 gene measured by western blot.

FIG. 12 shows the changes in collagen deposition and Gtse1 protein expression level by the administration of mannose-modified lipid nanoparticles in the bleomycin-induced pulmonary fibrosis animal model.

FIG. 13 shows the changes in collagen deposition and Gtse1 protein expression level by the administration of mannose-modified lipid nanoparticles in a radiation-induced pulmonary fibrosis animal model.

FIG. 14 shows the histopathological changes and lung volume by the administration of mannose-modified lipid nanoparticles in the radiation-induced pulmonary fibrosis animal model.

FIG. 15 is a graph showing the lung capacity indices measured using Flexivent® in the radiation-induced pulmonary fibrosis animal model.

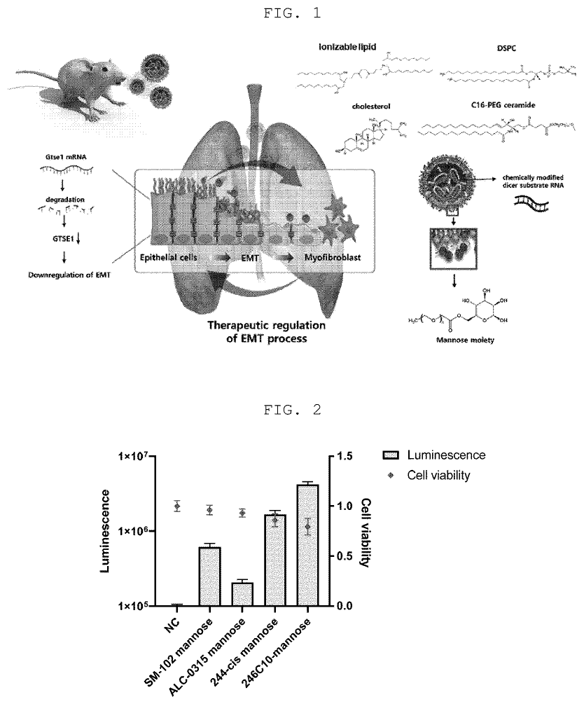

### BEST MODE

Hereinafter, the constitution and effects of the present invention will be described in more detail through the following Examples. These Examples are only provided for illustrating the present invention, but the scope of the present invention is not limited by these Examples.

### Example 1. Preparation of Lipid Nanoparticles

### Example 1-1. Preparation of Mannose-Modified Lipid Nanoparticles

Ionizable lipid (246-C10), cholesterol (Cholesterol powder, BioReagent, suitable for cell culture, ≥99%, sigma, Korea), phospholipid (DSPC; Avanti, USA), PEG-lipid conjugate (C16-PEG2000 ceramide or ceramide-PEG conjugate; C16 PEG2000 Ceramide, Avanti, USA) and mannose-PEG-lipid conjugate (Mannose-PEG-DSPE conjugate; Biochempeg, USA) were dissolved in ethanol at a molar ratio of 42.5:13:43 to 39.5:0.5:1.0 to 4.5 (wherein the amounts of cholesterol and total PEG-lipid conjugate (=PEG-ceramide conjugate amount+mannose-PEG-DSPE conjugate amount) were adjusted to ensure that the total molar ratio reached 100). The ethanol containing the ionizable lipid, cholesterol, phospholipid, PEG-ceramide conjugate, and mannose-PEG-lipid conjugate dissolved therein and acetate buffer were mixed at a volume ratio of 1:3 at a flow rate of 12 ml/min using a microfluidic mixing device (Benchtop Nanoassemblr™; PNI, Canada) to manufacture mannose-modified lipid nanoparticles (hereinafter referred to as ‘mannose-LNPs’).

### Example 1-2. Preparation of Mannose-Modified Lipid Nanoparticles Encapsulating Nucleic Acid

SM-102, ALC-0315, 246-C10, and 244-cis were prepared as ionizable lipids, and 244-cis was synthesized by the following Reaction Scheme:

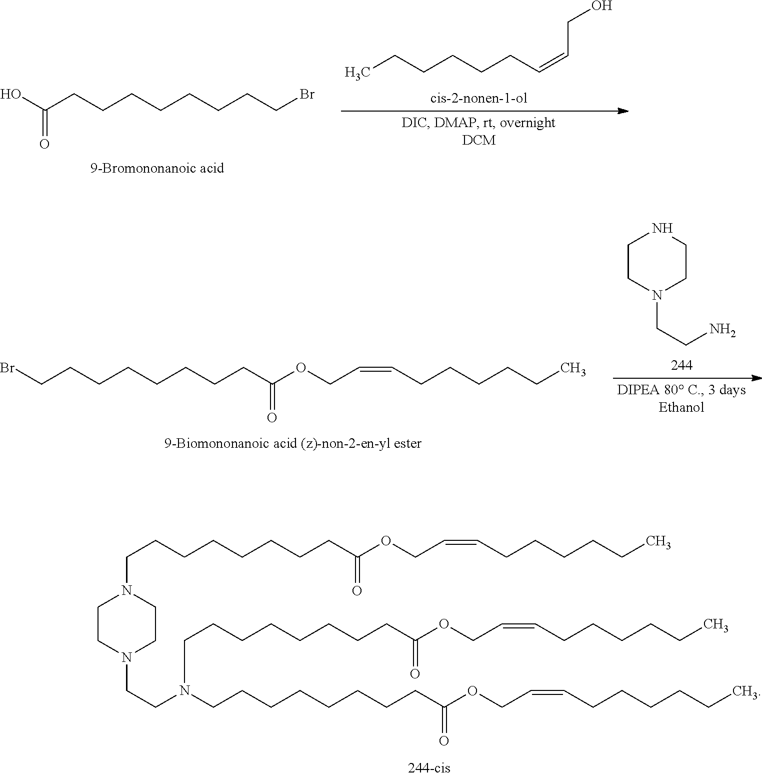

The organic phase (ethanol) containing the ionizable lipid, cholesterol, phospholipid, PEG-lipid conjugate (C16-PEG2000 ceramide), and mannose-PEG-lipid conjugate (mannose-PEG-DSPE conjugate) dissolved therein and the aqueous phase (sodium acetate or sodium citrate) containing the nucleic acid (mRNA or siRNA) dissolved therein were mixed at a flow rate of 12 mL/min using a microfluidic mixing device (Benchtop Nanoassemblr; PNI, Canada), and nucleic acids-encapsulated lipid nanoparticles were prepared specifically by the following method:
(i) in order to prepare mannose-modified lipid nanoparticles encapsulating mRNA, ionizable lipid phospholipid (DOPE or DSPC):cholesterol:C16-PEG2000 ceramide (PEG-ceramide conjugate), 1,2-dimyristoyl-rac-glycero-3-methoxypolyethylene glycol-2000 (DMG-PEG) or methoxypolyethyleneglycoloxy(2000)-N,N-ditetradecylacetamide (ALC-0159):mannose-PEG-lipid conjugate (mannose-PEG-DSPE conjugate) were dissolved in ethanol at a molar ratio of 26.5 to 50:10 to 20:38.5 to 52:0.5:1.0 to 4.5 (wherein the amounts of cholesterol and total PEG-lipid conjugate (=PEG-ceramide conjugate amount+mannose-PEG-DSPE conjugate amount) were adjusted to ensure that the total molar ratio reached 100). Then, the organic phase and the aqueous phase were mixed to reach the weight ratio of mRNA (luciferase mRNA; SEQ ID NO: 1 or Cre; SEQ ID NO: 2):ionizable lipid to 1:10, thereby preparing the lipid nanoparticles, and
(ii) in order to prepare mannose-modified lipid nanoparticles encapsulating siRNA, ionizable lipid (246-C10):phospholipid (DSPC):cholesterol:C16-PEG2000 ceramide:mannose-PEG-DSPE conjugate were dissolved in ethanol at a molar ratio of 42.5:13:43 to 39.5:0.5:1.0 to 4.5 (wherein the amounts of cholesterol and total PEG-lipid (=PEG-ceramide conjugate amount+mannose-PEG-DSPE conjugate amount) were adjusted to ensure that the total molar ratio reached 100). Then, the organic phase and the aqueous phase were mixed to reach the weight ratio of siRNA (siGtse1; SEQ ID NOs: 3 and 4):ionizable lipid to 1:7.5, thereby preparing the lipid nanoparticles.

The prepared lipid nanoparticles were dialyzed against PBS for 16 hours using a 3500 MWCO dialysis cassette to remove ethanol and adjust the pH of the lipid nanoparticles to that of the body.

### Example 2. Confirmation of Nucleic Acid Delivery Effect of Mannose-Modified Lipid Nanoparticles Depending on the Type of Ionizable Lipid

One day before transfection of cells with mannose-modified lipid nanoparticles, HeLa cells (Korea Cell Line Bank) were plated at 0.01×10⁶ cells/well in a white plate (96 well) and cultured in DMEM media (SH30022, Hyclone, USA) at 37° C. and 0.5-3% CO₂. The mannose-LNPs encapsulating luciferase mRNA prepared in Example 1-2 were stirred by pipetting, incubated at room temperature for 10 minutes, and then treated on HeLa cells (20 ng/well based on mRNA contained in lipid nanoparticles).

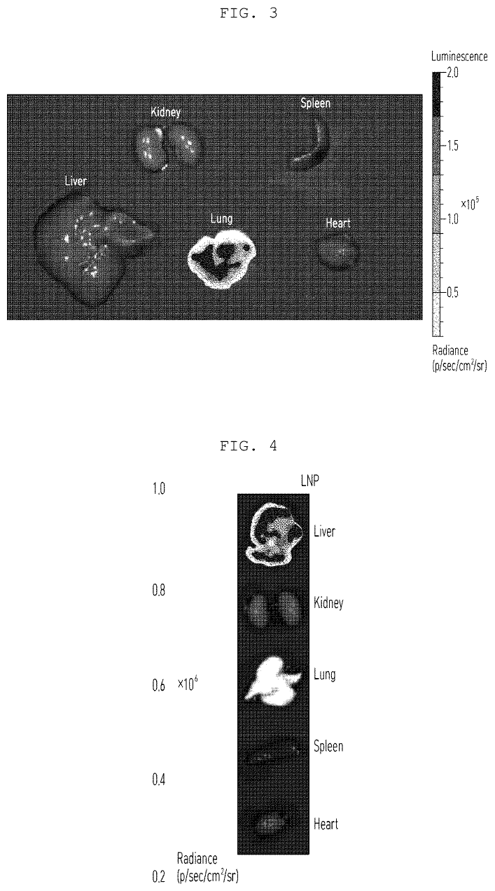

After 24 hours of treatment, the cells were treated with 100 μl/well of Bright-Glo™ Luciferase Assay solution (promega, USA) and left at room temperature for 10 minutes. Luminescence intensity of the lysed cells was measured using an Infinite M200 microplate luminometer (Tecan, USA), and the results are shown in FIG. 2.

It was confirmed from FIG. 2 that the mannose-modified lipid nanoparticles showed high luminescence values within cells regardless of the type of ionizable lipid.

### Example 3. Confirmation of Lung-Specific Delivery of Mannose-Modified Lipid Nanoparticles

### Example 3-1. Confirmation of Lung-Specific Delivery Using fLuc mRNA

The mannose-LNPs encapsulating luciferase mRNA prepared in Example 1-2 were injected intratracheally into the kidney, spleen, liver, lung, and heart of 7-week-old C57BL/6 mice at a dose of 0.1 mg/kg based on mRNA. After 3 hours, luciferin was administered intraperitoneally at a dose of 0.25 mg/kg, and bioluminescence was subsequently confirmed via ex vivo organ image using an IVIS system (PerkinElmer, USA), and the results are shown in FIG. 3 and Table 1.

TABLE 1

| sample | Mannose LNP |
|---|---|
| Hit | 1.965 × 10⁶ |
| background | 1.964 × 10³ |
| Hit/background | 1000 |
| Size (nm) | 92.4 |
| PDI | 0.124 |
| ee | 97.2 |
| (%) | |

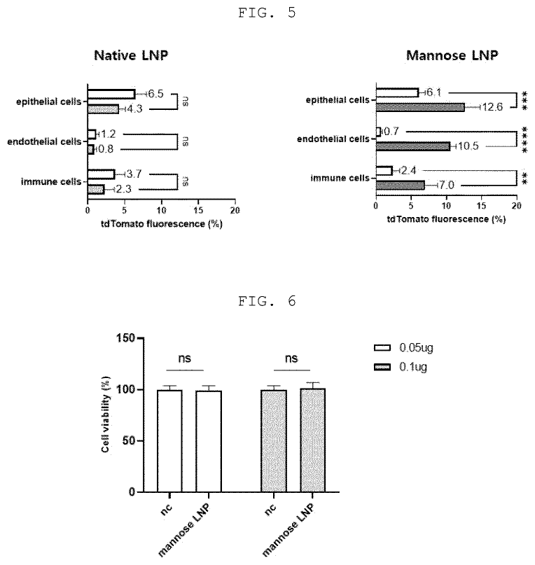

In addition, luc mRNA-encapsulated lipid nanoparticles (containing 1.5 mol % of lipid-PEG) were prepared in the same manner as in Example 1-2, except that the lipid nanoparticles were mannose-unmodified LNPs that did not contain mannose-PEG-lipid conjugate. The mannose-unmodified LNPs were intravenously injected into 7-week-old C57BL/6 female mice (Orient Bio) at a dose of 0.1 mg/kg based on mRNA. After 3 hours, luciferin was administered intraperitoneally at a dose of 0.25 mg/kg. Then, the mice were sacrificed, organs were extracted, and bioluminescence in each organ was subsequently confirmed via ex vivo organ image using an IVIS system, and the results are shown in FIG. 4.

As confirmed in FIGS. 3 and 4, mannose-LNPs showed high luminescence intensity in the lungs, whereas mannose-unmodified LNPs showed high luminescence intensity in the liver, confirming that the formulation of the lipid nanoparticle of the present invention was modified with mannose to be delivered specifically to the lungs.

### Example 3-2. Confirmation of Lung Cell-Specific Delivery Using Cre mRNA

The mannose-LNPs encapsulating Cre mRNA (mCre) prepared in Example 1-2 and the mannose-unmodified LNPs (Native LNPs) were administered intratracheally at a dose of 0.3 mg/kg based on mRNA to LSL-tdTomato mice with pulmonary fibrosis and LSL-tdTomato mice without pulmonary fibrosis, respectively. The mice were sacrificed, and the lungs were extracted. tdTomato fluorescence expression in each lung cell (epithelial cells, endothelial cells, immune cells) was confirmed using flow cytometry (LSRFortessa, BD). The results are shown in FIG. 5 (blank: normal model, colored: pulmonary fibrosis model).

As confirmed in FIG. 5, it was confirmed that the mannose-LNPs of the present invention were specifically delivered to fibrotic lungs compared to mannose-unmodified LNPs. In addition, the mannose-LNPs of the present invention showed different fluorescence expression patterns per lung cell compared to the mannose-unmodified LNPs, particularly confirming that the mannose-LNPs of the present invention were delivered most specifically to epithelial cells of fibrotic lungs.

### Example 4. Confirmation of Toxicity of Mannose-Modified Lipid Nanoparticles

### Example 4-1. Confirmation of Cytotoxicity

Whether the mannose-LNPs of the present invention exhibit cytotoxicity was confirmed through the CellTiter-Fluor™ Cell Viability Assay, which is an analysis method that confirms cell viability through fluorescence analysis of fluorescent AFC formed by cleavage of cell membrane-penetrating substrate by protease inside the cell.

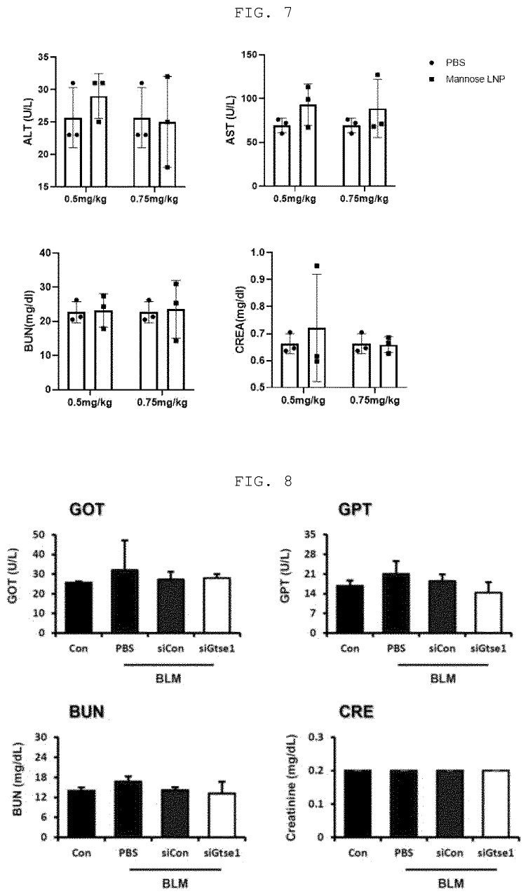

Specifically, L132 and MEF cells were seeded at a density of 0.1×10⁵ cells in a transparent 96 well plate (SPL, 96 Well Cell Culture Plates, 30096). After 24 hours, the lipid nanoparticles prepared in Example 1-1 were treated into the medium so that the total amount of lipid nanoparticles corresponding to 0.05 μg or 0.1 μg based on mRNA per well was 100 μl. After 24 hours of lipid nanoparticle treatment, 100 μl of CellTiter-Fluor™ Cell Viability Assay (Promega, Cat. #G6080) was added per well and the cells were incubated for 30 minutes. The absorbance at 450 nm was measured using Infinite® 200 PRO NanoQ (Tecan), and the results are shown in FIG. 6.

As confirmed in FIG. 6, the mannose-modified lipid nanoparticles of the present invention did not exhibit cytotoxicity.

### Example 4-2. Confirmation of Liver and Kidney Toxicity

To evaluate the presence of liver and kidney toxicity following the administration of mannose-LNPs of the present invention, the levels of aspartate aminotransferase (AST), alanine aminotransferase (ALT), blood urea nitrogen (BUN), and Creatinine were measured, respectively.

Specifically, the lipid nanoparticles prepared in Examples 1-2 were administered intratracheally once to 7-week-old C57BL/6 mice at doses of 0.5 and 0.75 mg/kg based on mRNA, and blood samples were collected 24 hours after administration to determine the levels of AST, ALT, BUN, and creatinine. The results are shown in FIG. 7.

As confirmed in FIG. 7, the mannose-modified lipid nanoparticles of the present invention did not exhibit significant liver and kidney toxicity.

### Example 4-3. Identification of Liver and Kidney Damage Parameters in Pulmonary Fibrosis Animal Model

The bleomycin-induced pulmonary fibrosis model was used to further determine whether administration of the mannose-LNPs of the present invention causes liver and kidney damage.

Specifically, 7-week-old C57BL/6 mice were intratracheally administered bleomycin at a dose of 1.8 mg/kg to establish a pulmonary fibrosis animal model, wherein 1 day before and 4 days after bleomycin administration, the mannose-modified lipid nanoparticles encapsulating siGtse1 prepared in Example 1-2 were intratracheally administered twice at a dose of 0.5 mg/kg based on siRNA. Then, on Day 14, the mice were sacrificed, the plasma was isolated from the collected blood, and the levels of glutamic oxaloacetic transaminase (GOT) and glutamic pyruvic transaminase (GPT) as liver damage parameters and BUN and creatinine (CRE) as kidney damage parameters were measured. The results are shown in FIG. 8 and Table 2.

TABLE 2

| | | Bleomycin (BLM) | | |
|---|---|---|---|---|
| Parameter | Con | PBS | siCon | siGtse1 |
| GOT (Glutamic Oxaloacetic Transaminase ) (U/L) | 25.7 ± 0.58 | 32 ± 15.25 | 27.2 ± 3.96 | 28 ± 2.00 |
| GPT (Glutamic Pyruvic Transaminase) (U/L) | 16.8 ± 1.89 | 21.0 ± 4.58 | 18.5 ± 2.38 | 14.3 ± 3.79 |
| BUN (Blood Urea Nitrogen) (mg/dL) | 14.0 ± 0.95 | 16.7 ± 1.59* | 14.2 ± 0.87 | 13.2 ± 3.53 |
| CRE (Creatinine) (mg/dL) | 0.2 ± 0.00 | 0.2 ± 0.00 | 0.2 ± 0.00 | 0.2 ± 0.00 |

As confirmed in FIG. 8 and Table 2, the mannose-modified lipid nanoparticles of the present invention did not exhibit significant liver and kidney toxicity.

### Example 5. Confirmation of Pathological Phenomena in Major Organs

To confirm the histopathological changes in major organs following administration of the mannose-LNPs of the present invention, the histopathological changes in the spleen, liver, kidney, and lungs were observed by hematoxylin-eosin (H&E) staining.

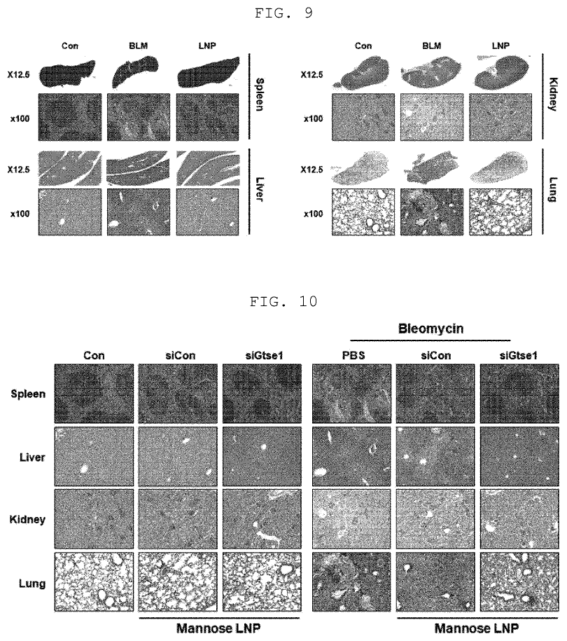

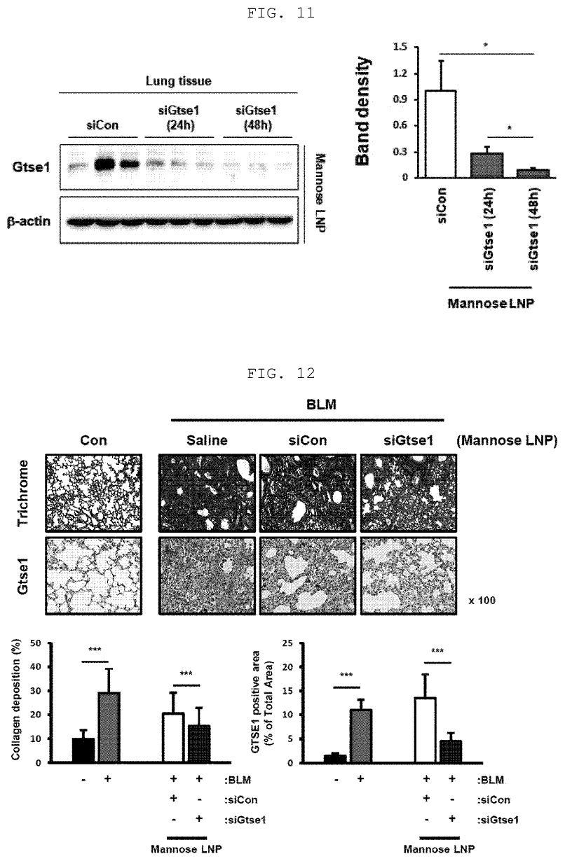

Specifically, 7-week-old C57BL/6 mice were administered bleomycin at a dose of 1.8 mg/kg, wherein 1 day before and 4 days after bleomycin administration, the mannose-modified lipid nanoparticles encapsulating siGtse1 prepared in Example 1-2 were intratracheally administered twice at a dose of 0.5 mg/kg based on siRNA. Then, on Day 14, the mice were sacrificed, the spleen, liver, kidneys, and lungs were stained with H&E, and the pathological phenomena were observed through an optical microscope, and the results are shown in FIGS. 9 and 10.

As confirmed in FIGS. 9 and 10, the bleomycin-administration group (BLM) showed damage to major organs, while no significant histopathologic changes were observed in mannose-LNP administration group.

### Example 6. Confirmation of Effect on Pulmonary Fibrosis

### Example 6-1. Silencing Effect on Pulmonary Fibrosis-Related Gene

The inhibitory effect of mannose-LNPs on the Gtse1 gene, which is known to be highly related to pulmonary fibrosis, was confirmed.

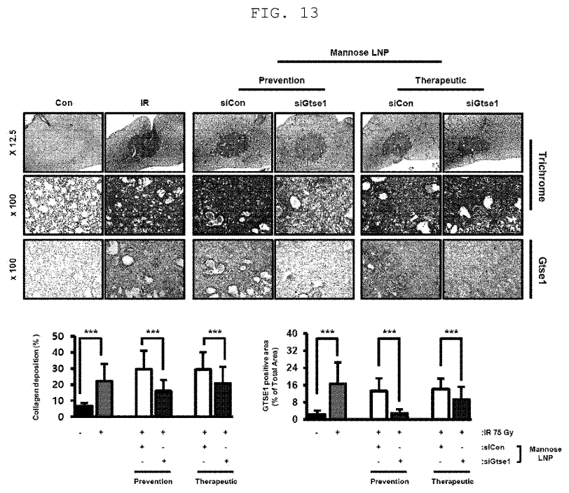

Specifically, the mannose-LNPs encapsulating siGtse1 prepared in Example 1-2 was intratracheally injected into 7-week-old C57BL/6 mice at a dose of 0.5 mg/kg based on siRNA. The mice were sacrificed after 24 and 48 hours, and Gtse1 protein in lung cells was measured by western blot. The results are shown in FIG. 11.

As confirmed in FIG. 11, the mannose-LNPs encapsulating siGtse1 could significantly inhibit the expression of Gtse1 protein in lung cells.

### Example 6-2. Confirmation of Effect in Bleomycin-Induced Pulmonary Fibrosis Animal Model

The effect of mannose-LNPs was confirmed using a bleomycin-induced pulmonary fibrosis animal model.

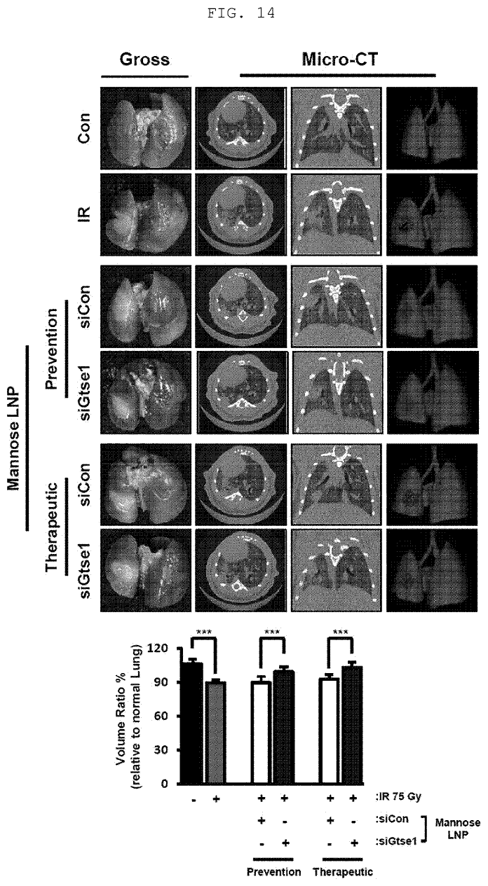

Specifically, 7-week-old C57BL/6 mice were intratracheally administered bleomycin at a dose of 1.8 mg/kg to establish a pulmonary fibrosis animal model, wherein 1 day before and 4 days after bleomycin administration, the mannose-modified lipid nanoparticles encapsulating siGtse1 prepared in Example 1-2 were intratracheally administered twice at a dose of 0.5 mg/kg based on siRNA. On Day 14, the mice were sacrificed and collagen deposition was confirmed through Masson's Trichrome staining, and the results are shown in FIG. 12.

As confirmed in FIG. 12, the bleomycin-administration group (Saline) and the mannose-LNP administration group (siCon) encapsulating siControl nucleic acid (Bioneer, Cat. #SN-1013) showed increased collagen deposition, whereas the mannose-LNP administration group (siGtse1) encapsulating siGtse1 nucleic acid drug showed a significant decrease in collagen, and immunohistochemistry staining also confirmed a decrease in the expression of Gtse1 protein.

### Example 6-3. Confirmation of Effect in Radiation-Induced Pulmonary Fibrosis Animal Model

The effect of mannose-LNPs was confirmed using a radiation-induced pulmonary fibrosis animal model.

Specifically, 10-week-old C57BL/6 mice were irradiated with a high-dose focal radiation of 75 Gy to a 3 mm volume of the left lung to establish a pulmonary fibrosis animal model. Then, the mannose-modified LNPs encapsulating siGtse1 prepared in Example 1-2 were administered intratracheally at a dose of 0.5 mg/kg based on siRNA, twice post-irradiation, on Day 1 and Week 3 for the prevention group and on Weeks 4 and 5 for the therapeutic group. On Week 6, the mice were sacrificed and collagen deposition was confirmed through Masson's Trichrome staining, and the results are shown in FIG. 13.

As confirmed in FIG. 13, both the prevention group and the therapeutic group administered mannose-LNPs encapsulating siGtse1 nucleic acid drug showed a significant decrease in collagen, and immunohistochemistry staining also confirmed a decrease in the expression of Gtse1 protein.

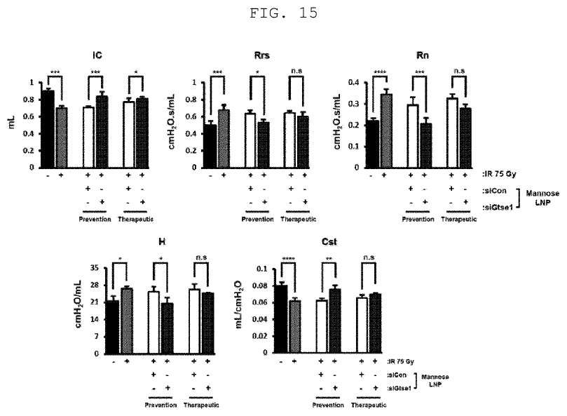

In addition, formalin liquid was inserted into the trachea of the sacrificed mouse to fix the entire lung, and histopathological changes in the irradiated area were examined visually. The extent of lung damage was assessed using small animal Micro-CT imaging and the results are shown in FIG. 14.

As confirmed in FIG. 14, both the prevention group and the therapeutic group administered mannose-LNPs encapsulating siGtse1 nucleic acid drug showed recovery of lung volume compared to the control group.

### Example 6-4. Verification of Various Lung Capacity Indices

It is known that progression of pulmonary fibrosis following radiation exposure leads to a decrease in lung volume, accompanied by changes in lung tissue elasticity and various lung capacity indices. Thus, the lung capacity indices in Table 3 below were measured in the animal model of Example 6-2 using Flexivent®, a test method that enables the measurement of various lung function tests measured in humans, in small animals, and the results are shown in FIG. 15.

TABLE 3

| Abbreviation | Parameter | Description | Obstructive | Restrictive |
|---|---|---|---|---|
| [symbol] | Respiratory | [symbol] | ↓ | [symbol] |
| | Capacity | [symbol] | | |
| [symbol] | Resistance of the | [symbol] | | ↑ |
| | respiratory system | [symbol] | | |
| | | [symbol] | | |
| R_{N} | Newtonian | [symbol] | | ↑ |
| | Resistance | [symbol] | | |
| | | [symbol] | | |
| [symbol] | [symbol] | [symbol] | ↓ | ↑ |
| | [symbol] | [symbol] | | |
| | | [symbol] | | |
| | | [symbol] | | |
| [symbol] | [symbol] | [symbol] | ↑ | ↓ |
| | [symbol] | | | |

[symbol]

indicates data missing or illegible when filed

As confirmed in FIG. 15, the irradiation group showed a restrictive trend in lung capacity indices compared to the control group, but the prevention group and the therapeutic group administered mannose-LNPs encapsulating siGtse1 nucleic acid drug showed a recovery in the restrictive trend in lung capacity indices compared to the irradiation group.

1. A composition for transpulmonary delivery of a drug comprising lipid nanoparticles containing a mannose-polyethyleneglycol (PEG)-lipid conjugate.

2. The composition for transpulmonary delivery of a drug of claim 1, wherein the composition is delivered locally into the lung.

3. The composition for transpulmonary delivery of a drug of claim 1, wherein the composition is delivered locally into the fibrotic lung.

4. The composition for transpulmonary delivery of a drug of claim 1, wherein the lipid nanoparticles further comprise at least any one selected from the group consisting of ionizable lipids, phospholipids, and structural lipids.

5. The composition for transpulmonary delivery of a drug of claim 4, wherein the ionizable lipid is at least any one selected from the group consisting of SM-102, ALC-0315, DODAC, DODMA, DSDMA, DOTMA, DDAB, DOTAP, DC-Chol, DMRIE, DOSPA, DOGS, CLinDMA, CpLinDMA, DMOBA, DOcarbDAP, DLinDAP, DLincarbDAP, DLinCDAP, DLinDMA, DLenDMA, DLin-MC3-DMA, 246-C10, and 244-cis.

6. The composition for transpulmonary delivery of a drug of claim 4, wherein the phospholipid is at least any one selected from the group consisting of DOPE, DSPC, POPC, EPC, DOPC, DPPC, DOPG, DPPG, DSPE, DOTAP, phosphatidylethanolamine, dipalmitoylphosphatidylethanolamine, 1,2-dioleoyl-sn-glycero-3-phosphate, 1,2-dilinoleoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine, POPE, DOPS, DLPC, DMPC, DUPC, 1,2-di-O-octadecenyl-sn-glycero-3-phosphocholine, 1-oleoyl-2-cholesterylhemisuccinoyl-sn-glycero-3-phosphocholine, 1-hexadecyl-sn-glycero-3-phosphocholine, 1,2-distearoyl-sn-glycero-3-phosphoethanolamine, 1,2-dilinoleoyl-sn-glycero-3-phosphoethanolamine, 1,2-dilinoleoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, and sphingomyelin.

7. The composition for transpulmonary delivery of a drug of claim 4, wherein the structural lipid is at least any one selected from the group consisting of cholesterol, cholestanol, spinasterol, fecosterol, sitosterol, ergosterol, ergostanol, campesterol, stigmasterol, brassicasterol, tomatidine, ursolic acid, and alpha-tocopherol.

8. The composition for transpulmonary delivery of a drug of claim 4, wherein the lipid nanoparticles comprise a mixture of the ionizable lipid:phospholipid:structural lipid:PEG-lipid conjugate in a molar ratio of 20 to 60 to 30:30 to 60:0.1 to 10.

9. A pharmaceutical composition for preventing or treating pulmonary diseases, comprising:
i) lipid nanoparticles containing a mannose-polyethyleneglycol (PEG)-lipid conjugate; and
ii) an anionic drug.

10. The pharmaceutical composition of claim 9, wherein the anionic drug is a nucleic acid drug.

11. The pharmaceutical composition of claim 10, wherein the nucleic acid drug is at least any one selected from the group consisting of siRNA, mRNA, antisense oligonucleotide, and miRNA.

12. The pharmaceutical composition of claim 9, wherein the pulmonary disease is pulmonary fibrosis.

13. The pharmaceutical composition of claim 9, wherein the composition is administered locally into the lung.


SEQUENCE LISTING (4 sequences)

SEQ ID NO: 1 - RNA, 1653 nt, synthetic construct
  source 1..1653: mol_type = other RNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 2 - RNA, 1074 nt, synthetic construct
  source 1..1074: mol_type = other RNA
atgggcacccccaagaagaagcggaaggtgcggaccgacctgaacagcaacctgctgaccgtgcaccagaacctgcccgccctgcccgtggacgccaccagcgacgaggtgcggaagaacctgatggacatgttccgggaccggcaggccttcagcgagcacacctggaagatgctgctgagcgtgtgccggagctgggccgcctggtgcaagctgaacaaccggaagtggttccccgccgagcccgaggacgtgcgggactacctgctgtacctgcaggcccggggcctggccgtgaagaccatccagcagcacctgggccagctgaacatgctgcaccggcggagcggcctgccccggcccagcgacagcaacgccgtgagcctggtgatgcggcggatccggaaggagaacgtggacgccggcgagcgggccaagcaggccctggccttcgagcggaccgacttcgaccaggtgcggagcctgatggagaacagcgaccggtgccaggacatccggaacctggccttcctgggcatcgcctacaacaccctgctgcggatcgccgagatcgcccggatccgggtgaaggacatcagccggaccgacggcggccggatgctgatccacatcggccggaccaagaccctggtgagcaccgccggcgtggagaaggccctgagcctgggcgtgaccaagctggtggagcggtggatcagcgtgagcggcgtggccgacgaccccaacaactacctgttctgccgggtgcggaagaacggcgtggccgcccccagcgccaccagccagctgagcacccgggccctggagggcatcttcgaggccacccaccggctgatctacggcgccaaggacgacagcggccagcggtacctggcctggagcggccacagcgcccgggtgggcgccgcccgggacatggcccgggccggcgtgagcatccccgagatcatgcaggccggcggctggaccaacgtgaacatcgtgatgaactacatccggaacctggacagcgagaccggcgccatggtgcggctgctggaggacggcgactga

SEQ ID NO: 3 - RNA, 25 nt, synthetic construct
  source 1..25: mol_type = other RNA
cagaagtcccatgtcacttatcacc

SEQ ID NO: 4 - RNA, 27 nt, synthetic construct
  source 1..27: mol_type = other RNA
acgtcttcagggtacagtgaatagtgg